(19) United States
(12) Patent Application Publication
(10) Pub. No.: US 2019/0135774 A1
(43) Pub. Date: May 9, 2019

(54) BIOMOLECULE PROCESSING FROM FIXED BIOLOGICAL SAMPLES

(71) Applicant: Cell Data Sciences, Inc., Fremont, CA (US)

(72) Inventor: Lucian Orbai, Oakland, CA (US)

(73) Assignee: Cell Data Sciences, Inc., Fremont, CA (US)

(21) Appl. No.: 16/095,346

(22) PCT Filed: Apr. 21, 2017
(86) PCT No.: PCT/US17/28849

§ 371 Date: Oct. 19, 2018

Related U.S. Application Data
(60) Provisional application No. 62/325,757, filed on Apr. 21, 2016.

(51) Int. Cl. C07D 313/02; C12Q 1/6806; C12N 15/10; C07D 307/34

(52) U.S. Cl. CPC C07D 313/02; C12Q 1/6806; C12Q 2523/101; C07D 307/34; C12N 15/1003

(57) ABSTRACT

Molecular characterization of disease has become the dominant trend in modern medicine, and it has recently become increasingly important to obtain qPCR, microarray, and next-generation sequencing (NGS) data for both research and clinical applications. Formalin-fixed, paraffin-embedded (“FFPE”) samples have become the standard way of storing clinical biopsies throughout the world. Unfortunately, the poor quality and quantity of nucleic acids obtained from such specimens has posed severe limitations on the types of studies that can be accomplished. Existing methods for retrieval of the biomolecules from their crosslinked matrices are poorly effective and rely on harsh conditions which further damage the biomolecules being extracted. The invention provides compositions and methods for retrieval, analysis and use of biomolecules, including nucleic acids, from such samples.

CROSS-REFERENCE

This application claims benefit under 35 U.S.C. § 119(e) to U.S. Provisional Application No. 62/325,757 filed Apr. 21, 2016, which is herein incorporated by reference in its entirety.

### BACKGROUND

Molecular characterization of disease has become the dominant trend in modern medicine, and it has recently become increasingly important to obtain qPCR, microarray, and next-generation sequencing (NGS) data for both research and clinical applications.

For example, in cancer, targeted approaches to diagnosis, prognosis, and treatment are becoming more and more important. Current clinical practice, especially in cancer but also other diseases, routinely relies on the use of tissue specimens isolated from patients in the form of biopsies or surgical specimens which are chemically fixed following isolation. Specimens are generally treated with a preservation solution containing a crosslinking agent, typically formaldehyde, in order to preserve tissue morphology and features. Following fixation, the specimens are encased in paraffin wax and stored for subsequent diagnosis and analysis. Such formalin-fixed, paraffin-embedded (“FFPE”) samples have become the standard way of storing clinical biopsies throughout the world. At least hundreds of millions of samples currently exist in the United States and elsewhere, and it is estimated that tens of millions of new specimens are generated every year. Tumor biopsy FFPE specimens are often accompanied by potentially very valuable information including cancer type and stage, patient survival, and treatment regime. Although this information could be correlated with expression and sequencing data, in practice the poor quality and quantity of nucleic acids obtained from such specimens has posed severe limitations on the types of studies that can be accomplished.

These methods, while generally effective for preservation of tissue structure for routine pathology, pose a significant challenge to isolation of biomolecules for molecular analysis. For instance, formaldehyde stabilizes the tissue for storage, but it also forms extensive crosslinks and adducts with the biomolecules in the sample, which strongly interferes with molecular analysis. Existing methods for retrieval of the biomolecules from their crosslinked matrices are poorly effective and rely on harsh conditions which further damage the biomolecules being extracted. This presents a formidable barrier for isolating, amplifying and sequencing biomolecules such as RNA and DNA from these patients, and therefore presents a large and growing problem for diagnosis and treatment of diseases including cancer.

### SUMMARY OF THE INVENTION

Provided herein are methods for isolating, purifying and/or analyzing biomolecules from biological samples with better yields, integrity and suitability in downstream applications. In some cases, a method is provided for removing formalin-induced chemical modifications from a nucleic acid, comprising incubating the nucleic acid with a solution comprising an uncrosslinking agent of Formula I or Formula II:

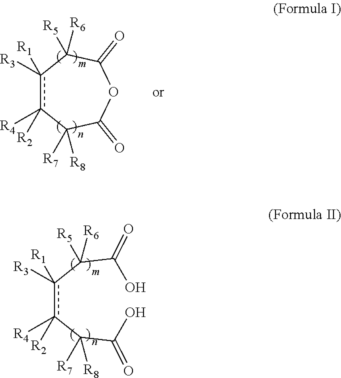

wherein:

m and n are independently 0 or 1;

when m is 1, R₅ and R₆ are independently —H or alkyl; when m is 0, R₅ and R₆ are absent;

when n is 1, R₇ and R₈ are independently —H or alkyl; when n is 0, R₇ and R₈ are absent;

R₁ and R₂ are independently —H, alkyl, —COOH, or halo; or R₁ and R₂ taken together form a five or six-membered cycloalkyl, heterocycloalkyl, or aryl ring;

when [symbol] is a single bond, R₃ and R₄ are independently -H or alkyl; when [symbol] is a double bond, R₃ and R₄ are absent.

In some cases, the uncrosslinking agent is not citric acid, trans-aconitic acid, 1,2,4-butanetricarboxylic acid, 1,4-cyclohexanedicarboxylic acid, 1,2,3,4,5,6-cyclohexanehexacarboxylic acid, isocitric acid, tricarballylic acid, succinic acid, or glutaric acid

For example, [symbol] is a double bond. Alternatively, [symbol] is a single bond.

In some instances, the uncrosslinking agent is a compound of Formula I:

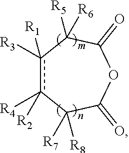

for example the uncrosslinking agent has the formula:

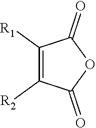

In other instances, the uncrosslinking agent is a compound of Formula II:

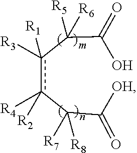

for example the uncrosslinking agent has the formula:

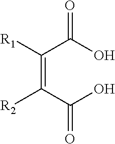

R₁ and R₂ are independently —H or —CH₃. In some cases, at least one of R₁ and R₂ is —CH₃. In some instances, R₁ and R₂ form a five or six-membered cycloalkyl or heterocycloalkyl ring.

In any of the formulas described herein, m and n are independently 0, 1. For example, m is 1 and n is 1, m is 0 and n is 0, m is 1 and n is 0, or m is 0 and n is 1.

Further provided herein is a method for removing formalin-induced chemical modifications from a nucleic acid, comprising incubating the nucleic acid with a solution comprising an uncrosslinking agent which is a polycarboxylic acid or polycarboxylic acid anhydride. In some cases, the uncrosslinking agent is not citric acid, trans-aconitic acid, 1,2,4-butanetricarboxylic acid, 1,4-cyclohexanedicarboxylic acid, 1,2,3,4,5,6-cyclohexanehexacarboxylic acid, isocitric acid, tricarballylic acid, succinic acid, or glutaric acid. In some cases, the polycarboxylic acid anhydride is not citraconic acid anhydride.

In some cases, the nucleic acid is present in a biological sample. For example, the biological sample is a formalin-fixed blood sample or a formalin-fixed, paraffin embedded (FFPE) tissue specimen.

In some instances, the method includes the step of heating the nucleic acid in the presence of the uncrosslinking agent at a temperature equal to or greater than 37° C., 50° C., 60° C., 65° C., or 70° C. For example, the heating is performed for at least 20 minutes, 30 minutes, 40 minutes, 1 hour, or 2 hours. In some instances, the heating is performed for at least 1 hour at a temperature above 65° C.

In some cases, the method further includes the step of treating the biological sample with a lysis solution comprising a buffering agent. The pH of the lysis solution is, for example, between about pH 5 and pH 9, between about pH 5.5 and about pH 8, or between about pH 6 and pH 7.5. The lysis solution may also comprise a chaotropic salt, for example a guanidinium salt including but not limited to guanidinium chloride or guanidinium isothiocyanate. In some cases, the lysis solution comprises a proteolytic enzyme. In some cases, the lysis solution comprises a detergent or surfactant.

The nucleic acid to be purified or analyzed is DNA or RNA, including but not limited to mRNA or non-coding RNAs. In some cases, the method comprises a further analysis step, such as sequencing the nucleic acid, or using the nucleic acid as a template in a nucleic acid amplification, including but not limited to PCR, for example RT-PCR.

The invention further provides kits comprising an uncrosslinking agent as described herein. The kits may further comprise a solid support for purifying a nucleic acid, such as a silica membrane, a silica spin column or silica micro- or nano-beads. Kits may also contain a proteolytic enzyme, for example Proteinase K. A kit may additionally comprise a user instruction manual. Such a user manual may instruct a user to perform a nucleic acid isolation or extraction, a nucleic acid amplification reaction, or nucleic acid sequencing. In some embodiments, a kit is provided which comprises: a) an uncrosslinking agent; b) a lysis solution comprising a buffer and a detergent; and c) optionally a proteolytic enzyme, for example Proteinase K.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1. Electropherogram of an RNA sample obtained using Qiagen RNEasy FFPE Kit.

FIG. 2. Electropherogram of an RNA sample obtained using an extraction method of the invention.

FIG. 3. Electropherogram of an RNA sample obtained using an extraction method of the invention.

### DETAILED DESCRIPTION

Provided herein are methods, compositions and devices for isolation, purification, and analysis of biomolecules from fixed tissue samples. The biomolecules obtained or analyzed through the taught methods have higher yield, quality, and/or better suitability in subsequent applications.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

As used herein the specification, “a” or “an” may mean one or more. As used herein in the claim(s), when used in conjunction with the word “comprising,” the words “a” or “an” may mean one or more than one.

The term “nucleic acid” or “polynucleotide” refers to deoxyribonucleic acids (DNA), ribonucleic acids (RNA). The term encompasses both single- or double-stranded forms, and includes nucleic acids which are naturally occurring as well as synthetically modified or produced. Unless specifically limited, the term encompasses nucleic acids containing known analogues of natural nucleotides that have similar binding properties as the reference nucleic acid.

The term “biomolecule” encompasses biological materials composed of proteins, nucleic acids, lipids, and carbohydrates, as well as aggregates, conjugates, or derivatives thereof. Biomolecules can be monomeric or polymeric, or can be structures within cells (organelles or fragments of organelles).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid. Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

The term “isolated,” when applied to a nucleic acid or protein, denotes that the nucleic acid or protein is substantially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. The nucleic acid or protein may be at least 85% pure, at least 95% pure, or at least 99% pure.

The term “alkyl,” by itself or as part of another substituent means, unless otherwise stated, a straight or branched chain, or cyclic hydrocarbon radical, or combination thereof, which may be fully saturated, mono- or polyunsaturated and can include di- and multivalent radicals, having between one and ten carbons, unless otherwise specified (e.g. C₁-C₁₂, meaning one to twelve carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”. The term “alkylene” by itself or as part of another substituent means a divalent radical derived from an alkane. The term further includes those groups described below as “heteroalkylene.” A “lower alkyl” or “lower alkylene” is a shorter chain alkyl or alkylene group, having six or fewer carbon atoms.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH=CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic, substituent that can be a single ring or multiple rings (preferably from 1 to 3 rings), which are fused together or linked covalently. The tam “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, tetrazolyl, benzo[b]furanyl, benzo[b]thienyl, 2,3-dihydrobenzo[1,4]dioxin-6-yl, benzo[1,3]dioxol-5-yl and 6-quinolyl. Substituents for each of the above noted aryl and heteroaryl ring systems are selected from the group of acceptable substituents described below.

The term “aryl”, when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, and the like).

Each of the above terms (e.g., “alkyl,” “heteroalkyl,” “aryl” and “heteroaryl”) is meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —CN and —NO₂ in a number ranging from zero to (2 m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” is meant to include oxygen (O), nitrogen (N), sulfur (S) and silicon (Si).

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The methods of the invention generally involve the isolation, whether complete or partial, of a biomolecule from a biological sample. The biological sample may be from a mammal, avian, amphibian, reptile, plant, bacteria, virus, or pathogen. The biological sample may comprise one or more cells derived from mammalian, avian, amphibian, reptilian, plant, bacterial or one or more pathogenically-infected cells. The mammal may be a human, goat, sheep, cow, pig, cat, dog, mouse, rat, or rabbit. In some embodiments, the samples are human samples.

In some cases, the samples are FFPE (formalin-fixed, paraffin embedded) samples. For examples, the samples are biopsies isolated from human patients suffering from a disorder. In some cases, the disorder is cancer. In other cases, the samples are blood samples which have been fixed using formalin. The term formalin, where used in the present specification, is intended to indicate an aqueous solution of formaldehyde.

Prior to analysis or extraction of a paraffin-embedded sample, it may be necessary to obtain thin sections of tissue, for example by using a microtome. The section may be at least 1, 3, 5, 10 or 20 μM thick, and the analys or extraction may use between 1 and 10, more preferably between 1 and 5 sections. In some cases, the section is processed by laser microdissection to isolate the desired components of the tissue, including but not limited to separation of tumor cells from surrounding normal tissue.

When the fixed tissue sample is embedded in a block of paraffin wax, it may be necessary to separate the tissue from the surrounding paraffin in order to further process the tissue. Removal of paraffin may be performed according to any method known in the art. One method of paraffin removal involves placing a tissue section in a solvent such as xylenes, thereby dissolving the paraffin surrounding the tissue. After centrifugation, the xylene supernatant is removed from the pellet containing the tissue. To remove residual xylenes, the pellet is washed with ethanol and then dried. Other solvents may be used instead of xylenes, for example terpene oils (e.g. D-limonene, sold under the trade name AmeriClear™, Cardinal Health, Inc., Dublin, Ohio), isoparaffinic hydrocarbons, vegetable oils (including, but not limited to coconut and olive oils). Surfactant and detergents may also be used in aqueous solution. Other methods of are known described, e.g. in US Application No. 2010/0075372; US Application No. 2006/0019332; Buesa et al. Ann Diagn Pathol. 2009; 13:246-256; and Chen et al. Biotech Histochem. 2010 August 85(4):231-40. In some cases, paraffin removal is effected by heating the section in an aqueous solution (such as a lysis buffer) above the melting point of paraffin, which then floats to the top of the sample and can be easily separated from the rest of the solution. In other cases, removal of paraffin can be effected manually, by carefully separating the tissue from the surrounding paraffin using a scalpel.

To separate the desired biomolecules from other cellular components, an aqueous lysis solution may be used. Generally, the lysis solution includes a buffering agent. For exmple, the buffering agent is Tris, MES, PIPES, MOPS, PIPES, HEPES, ADA, ACES, MOPSO, cholamine chloride, BES, TES, DIPSO, acetamidoglycine, TAPSO, POPSO, HEPPSO, HEPPS, tricine, glycinamide, bicine, TAPS, citrate, acetate, or phosphate. In some embodiments, the buffering agent is Tris. In some embodiments, the buffering agent is citrate. In some embodiments, the buffering agent is MES. In some embodiments, the buffering agent is PIPES. In some embodiments, the buffering agent is HEPES.

The buffering agent may be present at any concentration suitable to maintain the pH within the desired range during the lysis reaction. In some cases, the buffering agent is present in the lysis buffer at a concentration range between about 1 and about 500 mM, for example between about 5 and about 500 mM, between about 10 and about 500 mM, between about 1 and about 200 mM, between about 5 and about 200 mM, between about 10 and about 200 mM, between about 1 and about 100 mM, between about 5 and about 100 mM, or between about 10 and about 100 mM.

The buffering agent will be used to maintain the pH of the solution between about pH 4 and about pH 9. In some cases, the pH of the solution is between about pH 5 and about pH 9, between about pH 6 and about pH 8.5, or between about pH 6 and pH 8.

In some embodiments, the lysis solution includes a chelating agent. In some embodiments, the chelating agent is ethylenediaminetetraacetic acid (EDTA). In other embodiments, the chelating agent is ethylene glycol tetraacetic acid (EGTA).

The lysis buffer may comprise a detergent and/or surfactant. In some cases, the detergent is a nonionic detergent or surfactant, including but not limited to Triton® X (e.g. Triton® X-100, Triton® X-100R, Triton® X-114), Tween® 20, Tween® 80, Nonidet P-40 (IGEPAL 630), Brij® (e.g. Brij® 35, 58, 93, S100, O20, S20, C10, O10, or S10), octyl glucoside, octyl thioglucoside, or a Pluronic® surfactant (e.g. Pluronic® F-108). In other cases, the detergent is an ionic detergent. Such detergents include sodium dodecyl sulfate (SDS), N-lauroyl sarcosine, cetyltrimethylammonium bromide (CTAB), or potassium oleate. In some embodiments, the detergent is a zwitterionic detergent, including but not limited to CHAPS, CHAPSO, or ZWITTERGENT® (e.g. ZWITTERGENT® 3-08, 3-10, 3-12, 3-14, or 3-16). The lysis buffer may comprise more than one detergent, and may comprise one or more detergents from different categories, such as a non-ionic detergent in combination with an ionic detergent. The detergent or surfactant may be present in an amount representing about 0.1% to about 5%, for example about 0.1% to about 2% w/vol of the lysis solution.

In some embodiments, the lysis buffer comprises a chaotropic salt. In some embodiments, the chaotropic salt is a guanidinium salt, such as guanidinium hydrochloride, guanidinium thiocyanate, or guanidinium isothiocyanate.

In some embodiments, the lysis solution comprises an RNAse inhibitor, which is a protein, protein fragment, peptide or small molecule which inhibits the activity of any or all of an RNAse, such as RNase A, RNase B, RNase C, RNase T1, RNase H, RNase P, RNAse I and RNAse III, RNAse inhibitors include, but are not limited to, ribonucleoside vanadyl complex (New Englad Biolabs, Ipswich, Mass.), ScriptGuard (Epicentre Biotechnologies, Madison, Wis.), Superase-in (Ambion, Austin, Tex.), Stop RNase Inhibitor (5 PRIME Inc, Gaithersburg, Md.), ANTI-RNase (Ambion), RNase Inhibitor (Cloned) (Ambion), RNaseOUT™ (Invitrogen, Carlsbad, Calif.), Ribonuclease Inhib III (Invitrogen), RNasin® (Promega, Madison, Wis.), Protector RNase Inhibitor (Roche Applied Science, Indianapolis, Ind.), Placental RNase Inhibitor (USB, Cleveland, OH) and ProtectRNA™ (Sigma, St Louis, Mo.).

The lysis solution may also comprise a proteolytic enzyme. In some embodiments, the proteolytic enzyme is trypsin, chymotrypsin, endoproteinase Asp-N, endoproteinase Arg-C, endoproteinase Glu-C (V8 protease), endoproteinase Lys-C, thermolysin, papain, proteinase K, subtilisin, clostripain, exopeptidase, carboxypeptidase A, carboxypeptidase B, carboxypeptidase P, carboxypeptidase Y, cathepsin C, acylamino-acid-releasing enzyme, or pyroglutamagte aminopeptidase. For instance, the proteolytic enzyme is proteinase K.

The biological sample may be incubated in the lysis solution at a certain temperature for a period of time. The temperature may be, for example, about 25° C. or above. The temperature may be about 30° C., about 37° C., about 40° C. or above. The temperature may be about 45° C. or above. Sometimes, the temperature may be between about 25° C. and about 100° C., about 30° C. and about 95° C., about 37° C. and about 90° C., about 40° C. and about 85° C., or about 45° C. and about 80° C. The temperature may be between about 50° C. and about 80° C. In some instances, the temperature is between about 50° C. and about 80° C. Sometimes, the temperature is between about 50° C. and about 60° C. Sometimes, the temperature is between about 60° C. and about 70° C. Sometimes, the temperature is between about 70° C. and about 80° C. The temperature may be at least above 50° C. The temperature may be at least 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., or above 79° C. The temperature may be at most 50° C., 51° C., 52° C., 53° C., 54° C., 55° C., 56° C., 57° C., 58° C., 59° C., 60° C., 61° C. 62° C., 63° C., 64° C., 65° C., 66° C., 67° C., 68° C., 69° C., 70° C., 71° C., 72° C., 73° C., 74° C., 75° C., 76° C., 77° C., 78° C., 79°, or less. In some instances, the temperature is about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. The biological sample may be incubated in the lysis buffer for at least 5 minutes, 15 minutes, 30 minutes, 1 hour, 1.5 hours, 2 hours or longer. In some cases, the biological sample may be incubated in the lysis buffer overnight.

The methods of the invention further include the use of an uncrosslinking agent which reverses at least some of the chemical modifications induced by formalin in biomolecules, such as crosslinks or chemical adducts. In some embodiments, the uncrosslinking agent is a carboxylic acid. For example, the carboxylic acid is a dicarboxylic or tetracarboxylic acid. In some embodiments, the uncrosslinking agent is not citric acid, trans-aconitic acid, 1,2,4-butanetricarboxylic acid, 1,4-cyclohexanedicarboxylic acid, 1,2,3,4,5,6-cyclohexanehexacarboxylic acid, isocitric acid, tricarballylic acid, succinic acid, and/or glutaric acid.

In some embodiments, the uncrosslinking agent is oxalic, malonic, succinic, glutaric, adipic, pimelic, suberic, azelaic, sebacic, fumaric, maleic, glutaconic, muconic, glutinic, citraconic, phthalic, or mesaconic acid. In some embodiments, the carboxylic acid is a substituted carboxylic acid. For example, the carboxylic acid is malic acid, aspartic acid, glutamic acid, tartronic acid, tartaric acid, diaminopimelic acid, saccharic acid, mesoxalic acid, oxaloacetic acid, or acetonedicarboxylic acid. An anhydride prepared from any of the carboxylic acids named herein may also be used. For example, the carboxylic acid anhydrides may be citric, succinic, maleic, citraconic, glutaric, phthalic, pyromellitic, naphthalic or diphenic anhydride.

In some embodiments, the uncrosslinking agent is a compound of Formula I or Formula II:

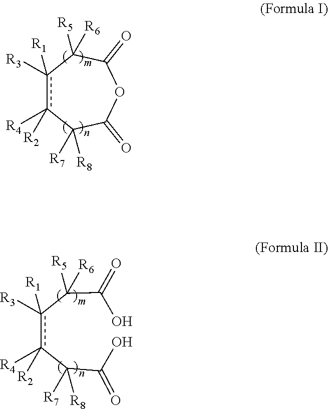

wherein:

m and n are independently 0 or 1;

when m is 1, R₅ and R₆ are independently —H or alkyl; when m is 0, R₅ and R₆ are absent;

when n is 1, R₇ and R₈ are independently -H or alkyl; when n is 0, R₇ and R₈ are absent;

R₁ and R₂ are independently —H, alkyl, —COOH, or halo; or R₁ and R₂ taken together form a five or six-membered cycloalkyl, heterocycloalkyl, or aryl ring;

when [symbol] is a single bond, R₃ and R₄ are independently —H or alkyl; when [symbol] is a double bond, R₃ and R₄ are absent;

with the proviso that the uncrosslinking agent is not citric acid.

In some cases, [symbol] is a double bond. In other cases, [symbol] is a single bond.

In some cases, the uncrosslinking agent is a compound of Formula Ia or IIa:

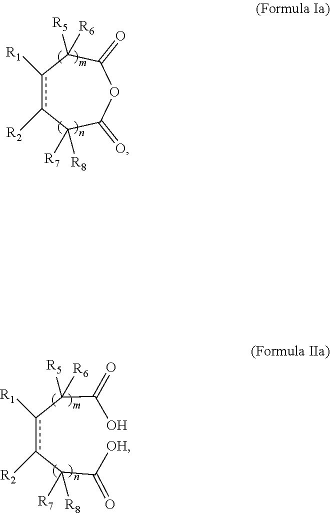

In some cases, the uncrosslinking agent is a compound of Formula I:

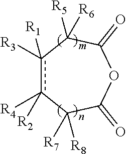

When the uncrosslinking agent is a compound of Formula I, m and n may be any combination of 0 and 1. For instance, both m and n are 0, m is 0 and n is 1, m is 1 and n is 0, or both m and n are 1. In some cases, m and n are 0.

In some cases, the uncrosslinking agent has the formula:

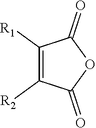

In some cases, the uncrosslinking agent is a compound of Formula II:

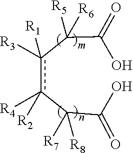

When the uncrosslinking agent is a compound of Formula II, m and n may be any combination of 0 and 1. For instance, both m and n are 0, m is 0 and n is 1, m is 1 and n is 0, or both m and n are 1. In some cases, m and n are 0.

In some cases, the uncrosslinking agent has the formula:

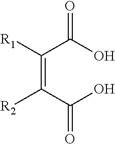

R₁ and R₂ are independently —H or —CH₃. In some cases, at least one of R₁ and R₂ is —CH₃. In some cases, R₁ is —CH₃ and R₂ is -H. In some cases, R₂ is —CH₃ and R₁ is —H.

In some cases, R₁ and R₂form a five, six, or seven-membered ring, for instance a cycloalkyl or heterocycloalkyl ring. In some cases, five or six-membered cycloalkyl or heterocycloalkyl ring.

When R₁ and R₂form a ring, the ring may be unsubstituted or substituted with a substituent as described herein.

In some cases, the uncrosslinking agent is maleic acid; citraconic acid; aconitic acid; cis-homoaconitic acid; 1-cyclopentene-1,2-dicarboxylic acid; 1-cyclohexene-1,2-dicarboxylic acid; 1-cycloheptene-1,2-dicarboxylic acid; 3-methyl-cyclopent-1-ene-1,2-dicarboxylic acid; 2,3-thiophenedicarboxylic acid; 1H-imidazole-4,5-dicarboxylic acid; 2-methyl-1H-imidazole-4,5-dicarboxylic acid; 2,5-dihydro-3,4-furandicarboxylic acid; 2-norbornene-2,3-dicarboxylic acid; oxabicyclo[2.2.1]hepta-2,5-diene-2,3-dicarboxylic acid; 2-methylbicyclo[2.2.2]oct-5-ene-2,3-dicarboxylic acid; 2-methylbicyclo[2.2.2]octane-2,3-dicarboxylic acid; 2-methyl-7-oxabicyclo[2.2.1]hept-5-ene-2,3-dicarboxylic acid; 2-methyl-7-oxabicyclo[2.2.1]heptane-2,3-dicarboxylic acid; or 2,3-dimethyl-7-oxabicyclo[2.2.1]heptane-2,3-dicarboxylic acid.

The biological sample may be treated with the uncrosslinking agent dissolved in a solvent, such as water, an aqueous solution, or any other solvent which is compatible with the biological sample. In some cases, the uncrosslinking agent is dissolved in a solvent used for deparaffinization. In other cases, the uncrosslinking agent is present in a buffered aqueous solution, such as a buffered lysis solution as described herein.

In some embodiments, the method of the invention involves treating the sample with an uncrosslinking agent at a temperature between about 40° C. and about 85° C., for example between about 50° C. and about 80° C., or between about 60° C. and about 80° C.

In some embodiments, the lysis solution comprises a compound comprising an amine. For instance, the compound is an amino acid, including but not limited to alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine. In other cases, the compound is an alkylamine, including but not limited to methylamine, ethylamine, propylamine, or ethylenediamine.

In some embodiments, the methods of the invention include the use of a compound which includes both an amine group and a proton-donating group. In some cases, the proton-donating group is a carboxylic acid, a phosphonic acid, or a sulfonic acid. In some cases, the proton-donating group is a phosphonic acid. In some cases, the compound is an aromatic compound in which the amine and the proton-donating group are substituted in an ortho configuration. In some cases, the compound is 2-aminobenzoic acid, (2-amino-5-methylphenyl)phosphonic acid, 2-aminobenzene-1-sulfonic acid, (2-hydrazinylphenyl) phosphonic acid, 6-amino-2H-1,3-benzodioxole-5-carboxylic acid, (6-amino-2H-1,3-benzodioxo1-5-yl) phosphonic acid, or 2-(1H-imidazol-4-yl)-4-methoxyaniline. In some embodiments, the catalyst is (2-amino-5-methylphenyl)phosphonic acid.

In other embodiments, the nucleic acids are further isolated and purified by the use of solution extraction or a solid support. For example, the solution extraction involves the use of phenol/chloroform or an alternative method such as TRIzol® (Chomczynski P, Sacchi N. Anal Biochem. 1987 April;162(1):156-9). In other instances, a solid support is used, such as a silica spin column or a silica-coated magnetic bead. Finely-milled ground glass, diatomaceous earths, or silica gels may also be used as solid supports.

In some embodiments, a nucleic acid obtained by the methods of the invention is a DNA molecule. The DNA molecule can be double-stranded or single-stranded.

In some embodiments, a nucleic acid obtained by the methods of the invention is an RNA molecule. The RNA molecule may be a small non-coding RNAs (ncRNAs). The small ncRNA may be a microRNA (miRNA), small interfering RNA (siRNA), trans-acting siRNA (tasiRNA); repeat-associated siRNA (rasiRNA); small hairpin RNA (shRNA), piwi-interacting RNA (piRNA), small nuclear RNA (snRNA), small nucleolar RNA (snoRNA), scan RNA (scnRNA), transcription initiation RNA (tiRNA), small modulatory RNA (smRNA), tiny non-coding RNA (tncRNA), QDE-2 interacting RNA (qiRNA), precursor miRNA (pre-miRNA), or short bacterial ncRNAs.

The overall integrity and quality of the extracted RNA can be calculated as the “RNA Integrity Number” or “RIN”, for example as measured using an Agilent® 2100 Bioanalyzer capillary electrophoresis instrument. See, e.g. Schroeder, A. et al., BMC. Mol. Biol. 7 (2006) 3; Imbeaud, S. et al. Nucl. Acids Res. (2005), 33, 6, e56, 1-12. In some embodiments, the RNA obtained using the methods of the invention has a RIN score of between 1 and 10, between 2 and 10, between 3 and 10, between 4 and 10, between 5 and 10, between 6 and 10. In some cases, the RIN score is greater than 1, 2, 3, 4, 5 or 6. In yet other cases, the RIN score is greater than 3. Alternatively, the RNA quality is measured as the ratio of amounts of 28S and 18S rRNAs.

Alternatively, the overall integrity and quality of the extracted RNA is calculated as a “DV200” value, representing the percentage of nucleic acids having a size greater than 200 nucleotides. See e.g. “Evaluating RNA Quality from FFPE Samples”, Technical Note: RNA sequencing, Illumina. Pub No. 470-2014-001, April 15 2014. A higher DV200 value is correlated with a better chance of success in subsequent applications such as RNA-seq or expression analysis. Generally, values greater than 70% indicate high quality samples; values between 50 and 70% indicate medium quality samples; values between 30 and 50% indicate low quality samples; and values lower than 30% are believed to be too degraded for downstream sequencing applications. In some embodiments, the RNA obtained by the method of the invention increases the DV200 by at least 3%, 5%, 10%, or 15%.

A nucleic acid molecule obtained as described herein may be subjected to various treatments, such as repair treatments and fragmenting treatments. Fragmenting treatments include mechanical, sonic, chemical, enzymatic, and degradation over time. Repair treatments include nick repair via extension and/or ligation, polishing to create blunt ends, removal of damaged bases including deaminated, derivatized, abasic, or crosslinked nucleotides. Nucleic acid molecules may also be subjected to chemical modification such as bisulfite conversion, methylation, demethylation, or extension.

In some cases, the nucleic acids obtained through the methods of the invention are used in a nucleic acid amplification reaction. As used herein, “nucleic acid amplification” refers to an enzymatic reaction in which the target nucleic acid is increased in copy number. Such increase may occur in a linear or in an exponential manner. For example, the nucleic acid amplification reaction is the polymerase chain reaction (“PCR”). In other instances, the nucleic acid amplification reaction is isothermal amplification. In yet other instances, the nucleic acid amplification reaction is rolling circle amplification. In further embodiments, nucleic acid amplification includes techniques such as nucleic acid sequence based amplification (NASBA), self-sustained sequence replication (3SR), loop mediated isothermal amplification (LAMP), strand displacement amplification (SDA), whole genome amplification, multiple displacement amplification, strand displacement amplification, helicase dependent amplification, nicking enzyme amplification reaction, recombinant polymerase amplification, reverse transcription PCR, ligation mediated PCR, or methylation specific PCR. In some embodiments, the nucleic acid amplification is performed quantitatively, such as in qPCR or RT-PCR.

In some embodiments, the nucleic acid amplification is used to detect an amplicon which is at least 50, 60, 70, 80, 100, 120, 150, 175, 200, 250, 300, 350, 400, or 500 bp long.

In some embodiments, the nucleic acids are used directly following the lysis step without prior purification of the nucleic acids obtained by the methods of the invention. For example, nucleic acid amplification is performed directly after the lysis step by adding the necessary reagents (including dNTPs and polymerase) to the lysis buffer.

In some embodiments, the nucleic acids obtained by the methods of the invention are used in sequencing, including next generation sequencing. Suitable sequencing techniques include Sanger sequencing, Illumina (Solexa) sequencing, pyrosequencing, next generation sequencing, Maxam-Gilbert sequencing, chain termination methods, shotgun sequencing, bridge PCR. Next generation sequencing methodologies may comprise massively parallel signature sequencing, polony sequencing, SOLiD sequencing, Ion Torrent semiconductor sequencing, DNA nanoball sequencing, Heliscope single molecule sequencing, single molecule real time (SMRT) sequencing. Other suitable sequencing techniques include nanopore DNA sequencing, tunnelling currents DNA sequencing, sequencing by hybridization, sequencing with mass spectrometry, microfluidic Sanger sequencing, microscopy-based techniques, RNA polymerase sequencing, in vitro virus high-throughput sequencing, or any other sequencing methodologies used in the art.

In some embodiments, the nucleic acid sequencing is whole genome sequencing (WGS), whole exome sequencing (WES), or transcriptome sequencing (e.g. RNA-Seq). In some applications, the RNA is enriched in target sequences. Such enrichment may be performed by capture of desired transcripts, by reverse transcription and amplification, or by depletion of non-target sequences such as ribosomal RNA. Exemplary commercial products for ribosomal RNA depletion include RiboZero (Epicentre/Illumina), e.g. as described in WO 2011/019993; RiboMinus (ThermoFisher Scientific); GeneRead (Qiagen); or the methods of WO 2014/044724.

In some embodiments, the methods of the invention are used to prepare a library for nucleic acid sequencing. In some cases, a nucleic acid library is prepared which is compatible with Illumina sequencing. Such libraries may be prepared, for example, using a Nextera™ DNA sample prep kit. Alternatively, a Tru-Seq sample prep kit is used. Additional approaches for generating Illumina next-generation sequencing library preparation are described, e.g., in Oyola et al. (2012). In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.). In some cases, nucleic acids are used to generate an Ion gDNA fragment library for the Ion Torrent System (ThermoFisher/Life Technologies). In this method, fragments of gDNA are ligated to adaptors, where at least one end of each fragment of genomic DNA is ligated to an adaptor including a barcode. The ligated adaptors and gDNA fragments may be nick repaired, size selected, and amplified using PCR with primers directed to the adaptors to produce an amplified library.

In some cases, the average sequence read length during Illumina sequencing is at least 100, 120, 150, 170 or 200 bp.

The invention further provides kits comprising an uncrosslinking agent as described herein. The kits may further comprise a solid support for purifying a nucleic acid, such as a silica membrane, a silica spin column or silica micro- or nano-beads. Kits may also contain a proteolytic enzyme, for example Proteinase K. A kit may additionally comprise a user instruction manual. Such a user manual may instruct a user to perform a nucleic acid isolation or extraction, a nucleic acid amplification reaction, or nucleic acid sequencing. In some embodiments, a kit is provided which comprises: a) an uncrosslinking agent; b) a lysis solution comprising a buffer and a detergent; and c) optionally a proteolytic enzyme, for example Proteinase K.

### EXAMPLES

### Example 1

Two extractions of total RNA (labeled “Extraction 1a” and “Extraction 1 b”) were performed according to the methods of the invention. Using a microtome, 10 μm thick sections were obtained from a formalin-fixed, paraffin embedded tissue block and transferred to a 1.5 mL Eppendorf® DNA/RNA LoBind tube. Each section was incubated in 40 μL 5 mM (2Z)-2-methylbut-2-enedioic acid. After incubating for 30 minutes at 72° C., the tube was cooled briefly on ice. 120 μL of Lysis Buffer (all buffers named herein refer to Cell Data Sciences RNAStorm™ Extraction Kit buffers) was added along with 3.2 units of Proteinase K (0.8 units/μL) and the sample was incubated at 56° C. for 15 minutes. The sample was subsequently heated to 72° C. for 2 hours. Following the lysis step, the contents of the sample were placed on ice for 5 minutes and spun down. The supernatant was transferred to a silica filter spin column and 160 μL of Binding Buffer were added, followed by 450 μL 200-proof ethanol. After washing 2× using Wash Buffer, RNA was eluted using 30 μL Elution Buffer.

For comparison purposes, a sample was also extracted from the same tissue block using the Qiagen RNEasy FFPE were processed according to the instructions provided by the manufacturer.

### Example 2

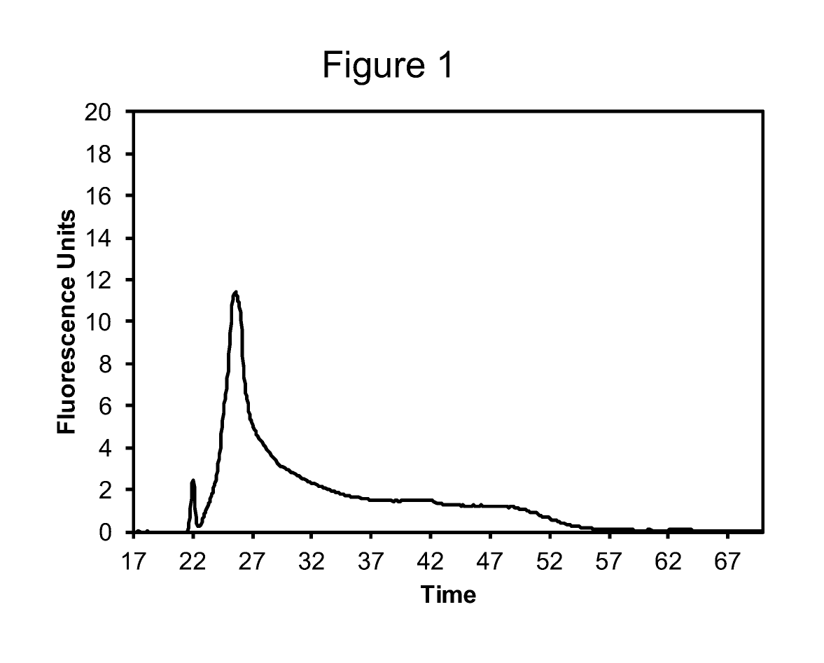

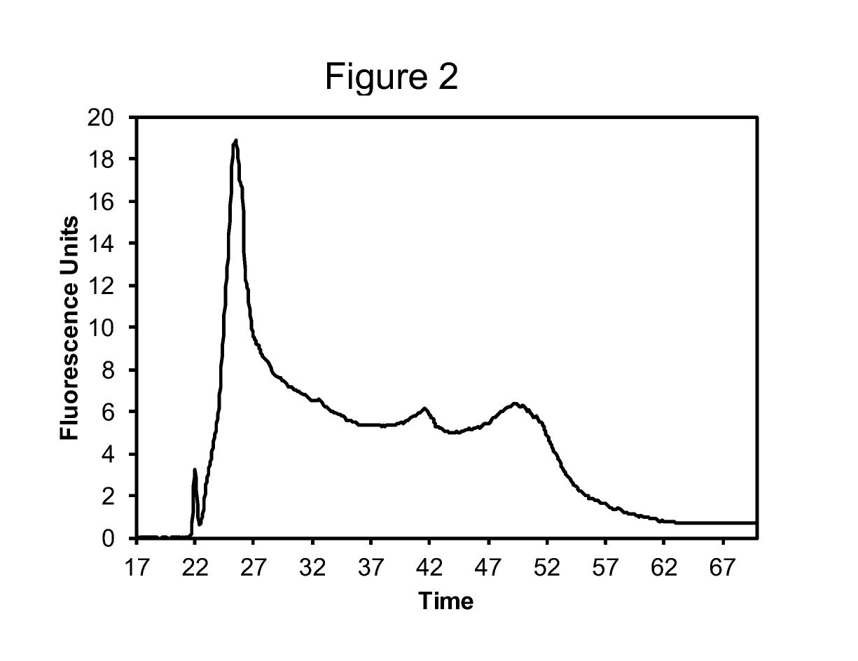

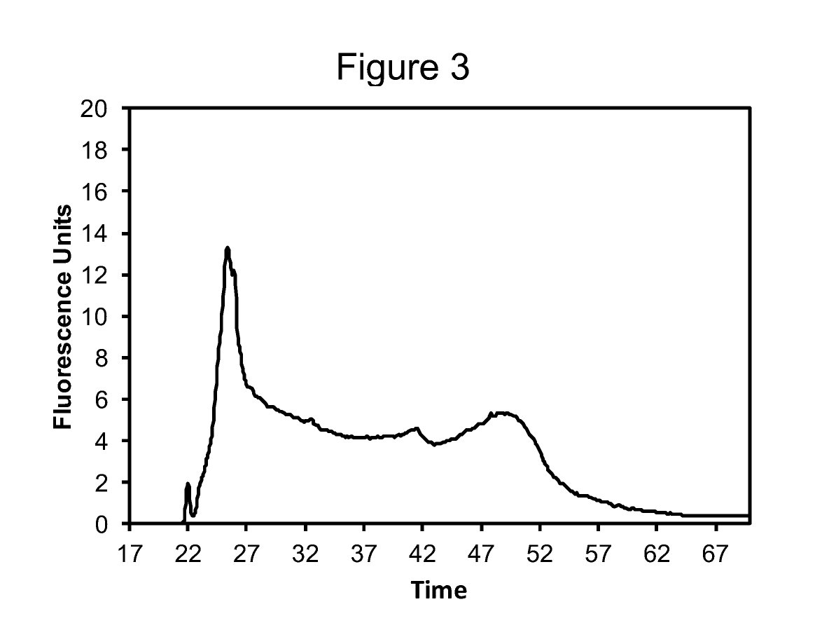

The RNA obtained according to the method of Example 1 was analyzed using capillary electrophoresis (Agilent BioAnalyzer 2100, RNA 6000 Pico Kit). Electropherograms are shown in FIGS. 1-3. The data for RNA obtained using the Qiagen RNEasy FFPE Kit is shown in FIG. 1. FIGS. 2 and 3 show data obtained for Extraction 1a and 1b, respectively.

### Example 3

The DV200 value was calculated for each extracted sample according to the method described by Illumina, Inc. See e.g. “Evaluating RNA Quality from FFPE Samples”, Technical Note: RNA sequencing, Illumina. Pub No. 470-2014-001, Apr. 15 2014. While other metrics have been used, such as yields as measured by Qubit or Nanodrop, or integrity values such as the Agilent RNA Integrity Number (“RIN”), these have not been found to work well with degraded FFPE samples. The DV200 value is believed to offer a much better overall indication of the quality of extracted nucleic acids.

Specifically, a DV200 value represents the percentage of nucleic acids having a size greater than 200 nucleotides. A higher DV200 value is correlated with a better chance of success in subsequent applications such as RNA-seq or expression analysis. Generally, values greater than 70% indicate high quality samples; values between 50 and 70% indicate medium quality samples; values between 30 and 50% indicate low quality samples; and values lower than 30% are believed to be too degraded for downstream sequencing applications.

The following DV200 values were observed. The RNA concentration of the eluted was also calculated for each sample using the Agilent Expert software:

| Sample | DV200 | RNA Concentration |
|---|---|---|
| Extraction 1a | 65% | 177.5 ng/μL |
| Extraction 1b | 68% | 134.5 ng/μL |
| Qiagen RNEasy FFPE | 51% | 68.4 ng/μL |

### Example 4

The RNA obtained according to the method of Example 1 was analyzed using RT-PCR. Reverse transcription was performed using a ProtoScript® II First Strand cDNA Synthesis Kit (New England Biolabs Inc., Ipswich, Mass.) according to the instructions provided by the manufacturer. A mixture of oligo-dT and random hexamer primers was used for cDNA synthesis. The resulting cDNA was quantitated using an Applied Biosystems 7300 RT-PCR instrument and using a Forget-Me-Not™ Master Mix (Biotium Inc., Hayward, Calif.). The primers used were designed to amplify a 514 bp amplicon within the GAPDH mRNA (forward primer sequence: 5′-CTGAACGGGAAGCTCACTGG-3′, reverse primer sequence: 5′-TGGTACATGACAAGGTGCGG-3′). Each RNA sample was analyzed in triplicate. The following Ct numbers were calculated using Applied Biosystems software:

| | Ct value (qPCR) | Standard Error |
|---|---|---|
| Extraction 1a | 31.3 | 0.04 |
| | 30.3 | 0.07 |
| | 29.45 | 0.02 |
| Extraction 1b | 30.7 | 0.04 |
| | 30.2 | 0.02 |
| | 30.5 | 0.04 |
| Qiagen RNEasy FFPE | 33.2 | 0.21 |
| | 34.6 | 0.09 |
| | 34.2 | 0.03 |

1. A method for removing formalin-induced chemical modifications from a nucleic acid, comprising incubating the nucleic acid with a solution comprising an uncrosslinking agent of Formula I or Formula II: 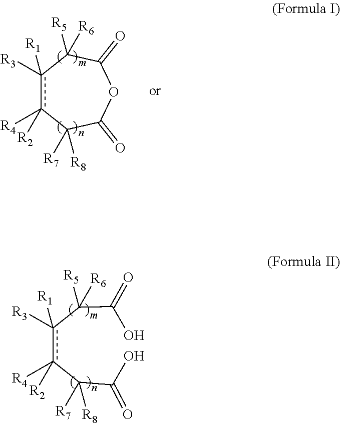
wherein:
m and n are independently 0 or 1;
when m is 1, R₅ and R₆ are independently —H or alkyl; when m is 0, R₅ and R₆ are absent;
when n is 1, R₇ and R₈ are independently —H or alkyl; when n is 0, R₇ and R₈ are absent;
R₁ and R₂ are independently —H, alkyl, —COOH, or halo; or R₁ and R₂ taken together form a five or six-membered cycloalkyl, heterocycloalkyl, or aryl ring;
when [symbol] is a single bond, R₃ and R₄ are independently -H or alkyl; when [symbol] is a double bond, R₃ and R₄ are absent;
with the proviso that the uncrosslinking agent is not citric acid, trans-aconitic acid, 1,2,4-butanetricarboxylic acid, 1,4-cyclohexanedicarboxylic acid, 1,2,3,4,5,6-cyclohexanehexacarboxylic acid, isocitric acid, tricarballylic acid, succinic acid, or glutaric acid.

2. The method of claim 1, wherein [symbol] is a double bond.

3. The method of claim 1, wherein the uncrosslinking agent is a compound of Formula I:

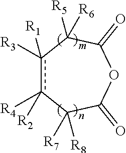

4. The method of claim 3, wherein the uncrosslinking agent has the formula:

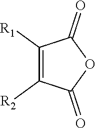

5. The method of claim 1, wherein the uncrosslinking agent is a compound of Formula II:

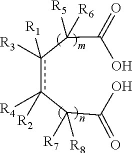

6. The method of claim 5, wherein the uncrosslinking agent has the formula:

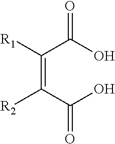

7. The method of claim 1, wherein R₁ and R₂ are independently —H or —CH₃.

8. The method of claim 7, wherein at least one of R₁ and R₂ is —CH₃.

9. The method of claim 1, wherein R₁ and R₂ form a five or six-membered cycloalkyl or heterocycloalkyl ring.

10. The method of claim 1, wherein m and n are 0.

11. (canceled)

12. (canceled)

13. The method of claim 1, wherein the nucleic acid is present in a biological sample.

14. The method of claim 12, wherein the biological sample is a formalin-fixed blood sample.

15. The method of claim 12, wherein the biological sample is a formalin-fixed, paraffin embedded (FFPE) tissue specimen.

16. The method of claim 1, wherein the method includes the step of heating the nucleic acid in the presence of the uncrosslinking agent at a temperature equal to or greater than 65° C.

17. The method of claim 16, wherein the heating is performed for at least 30 minutes.

18. The method of claim 17, wherein the heating is performed for at least 1 hour at a temperature above 65° C.

19. The method of claim 12, wherein the method further includes the step of treating the biological sample with a lysis solution comprising a buffering agent, and wherein wherein the pH of the lysis solution is between about pH 5 and pH 9.

20. (canceled)

21. (canceled)

22. (canceled)

23. The method of claim 19, wherein the lysis solution comprises a proteolytic enzyme.

24. The method of claim 19, wherein the lysis solution comprises a detergent or surfactant.

25. The method of claim 1, wherein the nucleic acid is DNA or RNA.

26.-32. (canceled)
